(19) United States
(12) Patent Application Publication
(10) Pub. No.: US 2008/0167531 A1
(43) Pub. Date: Jul. 10, 2008

(54) SYSTEM AND METHOD TO MONITOR BATTERY STATUS IN AN IMPLANTABLE MEDICAL DEVICE

(75) Inventor: Wesley McDermott, Shoreview, MN (US)

Correspondence Address: SCHWEGMAN, LUNDBERG & WOESSNER, P.A., P.O. BOX 2938, MINNEAPOLIS, MN, 55402, US

(73) Assignee: CARDIAC PACEMAKERS, INC., ST. PAUL, MN (US)

(21) Appl. No.: 11/620,513

(22) Filed: Jan. 5, 2007

(51) Int. Cl. A61B 5/00

(52) U.S. Cl. 600/300

(57) ABSTRACT

An implantable medical device includes a control circuit. A rechargeable battery is coupled to the control circuit. A battery monitoring circuit is coupled to one or more of the rechargeable battery and the control circuit. A telemetry circuit, coupled to the control circuit, is configured to transmit a battery status of the rechargeable battery to a first external device at a first time and to a second external device, of a different type than the first external device, at a second time.

### TECHNICAL FIELD

Various examples relate to the field of implantable medical devices, and in an example, but not by way of limitation, to a system and method to monitor a battery in an implantable medical device.

### BACKGROUND

Implantable medical devices include a built in power supply such as a battery. Such a battery has a limited life, and must be replaced or recharged at some point in time. If it is to be replaced, explant of the implantable device is required. If a battery is rechargeable, the recharging can be performed without an explant of the device via transcutaneous power transfer.

### OVERVIEW

An implantable medical device can include a control circuit, a rechargeable battery that is connected to the control circuit, a battery monitoring circuit that is connected to the rechargeable battery and/or the control circuit, and a telemetry circuit that is connected to the control circuit. The telemetry circuit can be configured to transmit a battery status of the rechargeable battery to a first external device at a first time and to a second external device at a second time. The second external device is of a different type than the first external device. In an example, the first external device can be a local external device to which a patient or a patient's local caregiver has access, and the second external device can be a remote patient monitoring system that is accessible by one or more of a physician and a plurality of implantable medical devices.

### BRIEF DESCRIPTION OF THE DRAWINGS

In the drawings, which are not necessarily drawn to scale, like numerals describe similar components throughout the several views. The drawings illustrate generally, by way of example, but not by way of limitation, various examples discussed in the present document.

FIG. 1 illustrates an example of an implanted medical device coupled to a first adjunct external device and a second adjunct external device.

FIG. 2 illustrates an example block diagram of an implantable medical device.

FIG. 3 illustrates a flowchart of an example process to monitor the status of a battery in an implantable medical device.

FIG. 4 illustrates a flowchart of another example process to monitor the status of a battery in an implantable medical device.

### DETAILED DESCRIPTION

The following detailed description refers to the accompanying drawings which form a part hereof, and in which is shown by way of illustration specific examples in which the disclosure may be practiced. These examples, which are sometimes referred to as examples, are discussed in sufficient detail to enable those skilled in the art to practice the teachings of the disclosure, and such examples may be combined, or other examples may be utilized, and structural, logical and electrical changes may be made without departing from the scope of the present disclosure. The following detailed description provides examples, and the scope of the present disclosure is defined by the appended claims and their equivalents.

It should be noted that references to “an”, “one”, or “various” examples in this disclosure are not necessarily to the same example, and such references contemplate more than one example. It should further be noted that references to “or” are meant to include “and”, “or”, and “and/or”.

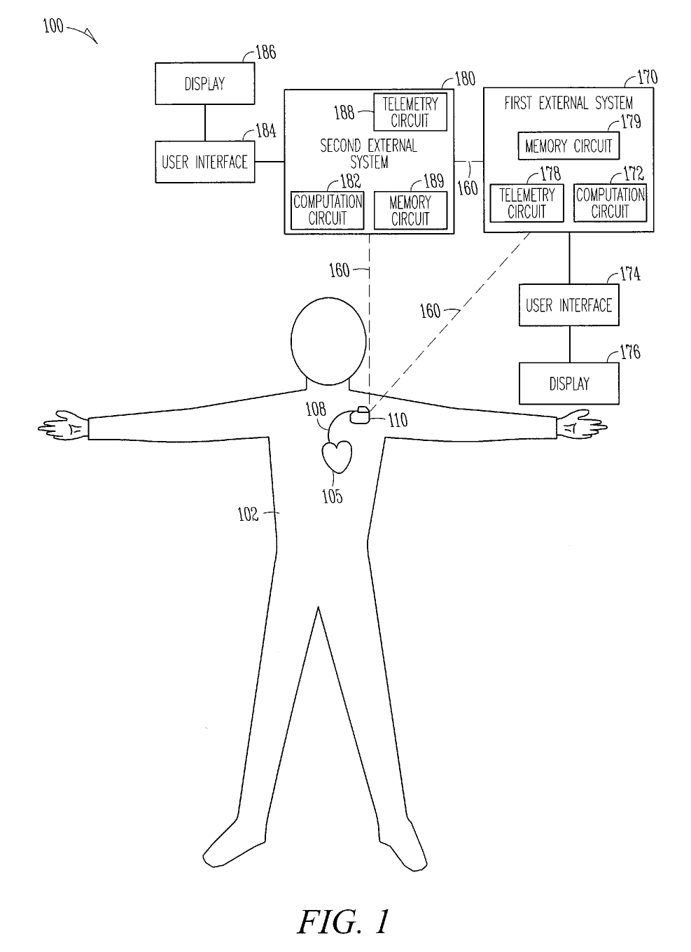

FIG. 1 illustrates an example of a medical device system 100, and portions of an environment in which it is used. The environment includes a body 102 with a heart 105. System 100 includes an implantable medical device 110, a lead system 108, a first adjunct device or system 170, a second adjunct device or system 180, and wireless telemetry links 160. The link between the first adjunct device and the second adjunct device may also be a wired connection. While FIG. 1 illustrates an implantable medical device in connection with a heart 105, the present disclosure is not so limited, and the present disclosure applies to any type of implantable medical device including, but not limited to, implantable pharmaceutical infusion pumps, implantable neural-stimulation devices, implantable hearing aids, inserted hearing aids, etc.

Data from the implantable device 110 may be transferred from the device 110 to the first external system 170 and the second external system 180 via the telemetry links 160. The data can be transferred directly to either of the external devices 170 or 180, or the data can be transferred from the implantable medical device 110 to the first external system 170, and the first external system 170 can then transfer that data to the second external system 180. The telemetered data loaded into the first external device 170 or the second external device 180 can then be used for analysis and interpretation either immediately or at a later time. In an example, the first external device 170 includes a local external programmer device, which allows a patient or a patient's aide to receive telemetered data from the implantable device 110 and also allows the patient or patient's aide to telemeter data or instructions from the local external programmer to the implantable device. In another example, the second external device 180 includes a remote external patient management device configured to manage implantable medical devices associated with different patients. A physician or other caregiver may use such a remote external patient management device to remotely monitor a patient with an implantable device. In another example, one or more local external repeaters couple the first external system 170 and the second external system 180.

FIG. 1 further illustrates that the first external device 170 can include a computation circuit 172 and the second external device 180 can include a computation circuit 182. The computation circuits 172 and 182 can compute information related to the status of the battery including one or more of a charge associated with the rechargeable battery, a voltage output of the rechargeable battery, a current flow through the rechargeable battery, and an amount of time that the rechargeable battery has been in use since implant or the last recharging of the battery. In an example, the computation circuit 182 in the second external device 180 can transmit information to a plurality of first external devices with which the second external device can communicate. As a result, the functionality of many first external devices can be altered simply by changing the configuration of the second external device. The first external device 170 can further include a user interface 174 that is connected to an external display 176, and the second external device 182 can further include a user interface 184 that is connected to an external display 186. In an example, the external displays 176 and 186 can be configured to display information relating to the status of a battery that supplies power to the implantable device 110. This status information can be displayed as a graphic. An example graphic is simple gauge, similar to a gas gauge in an automobile, and the gauge can include markings such as an Elective Replacement Indicator (ERI) or an End of Life (EOL) indicator. Additionally, the first external device 170 can include a telemetry circuit 178 and the second external device can include a telemetry circuit 188. The first external device 170 further can include a memory circuit 179 and the second external device can include a memory circuit 189.

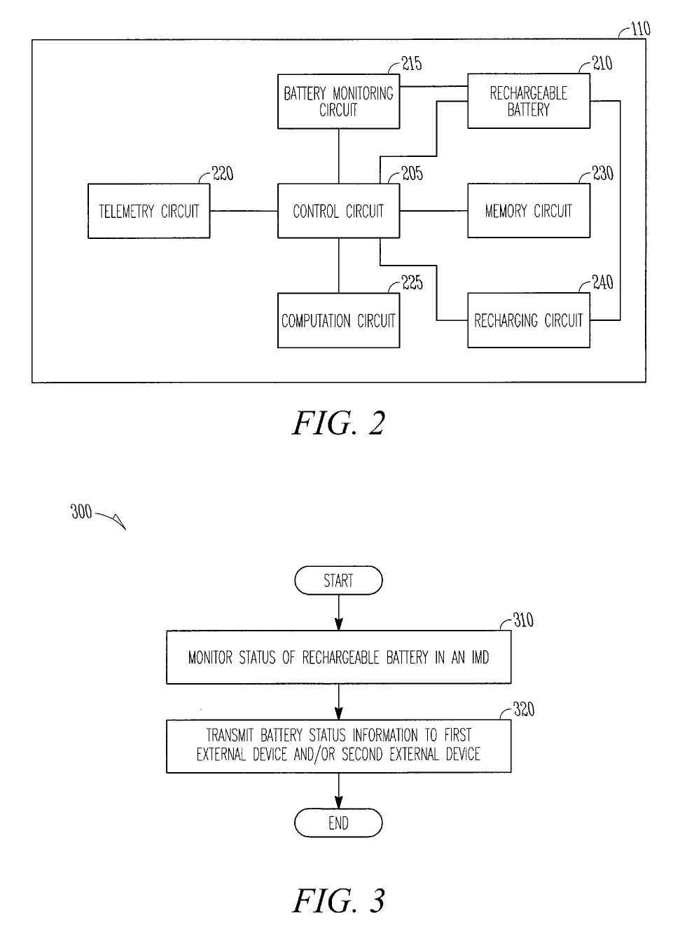

FIG. 2 illustrates an example of the implantable medical device 110 of FIG. 1. The implantable medical device includes a control circuit 205. Connected to the control circuit 205 are a rechargeable battery 210, a battery monitoring circuit 215, and a telemetry circuit 220. The battery monitoring circuit 215 can also be coupled to the rechargeable battery 210. FIG. 2 also illustrates that the implantable medical device 110 can have a computation circuit 225 coupled to the control circuit 205. Like the computation circuits 172 and 182 of the first and second external devices respectively, the computation circuit 225 can be configured to carry out computations relating to the status of the battery 210. Further, like the computation circuits 172 and 182, the computation circuit 225 can determine one or more of a remaining or delivered charge associated with the rechargeable battery, a voltage output of the rechargeable battery, a current flow through the rechargeable battery, and an amount of time that the rechargeable battery has been in use since implant or since the last recharging of the battery. The implantable medical device 110 may further include a memory circuit 230 coupled to the control circuit 205. The implantable medical device 110 can further include a recharging circuit 240 that is coupled to the control circuit 205 and the rechargeable battery 210.

The telemetry circuit 220 can be configured to transmit a battery status of the rechargeable battery 210 to the first external device 170 at a first time and to the second external device 180 at a second time. In an example, the first external device 170 is a different type of device than the second external device 180. In this manner, initial warnings about a waning charge of the rechargeable battery 210 can first be transmitted to a device that is accessible to and monitored by a patient with the implantable medical device. Then, if the battery monitoring circuit 215 determines that the remaining charge of the battery 210 has further decreased because the patient has failed to recharge the battery, another alert can be transmitted to the second external device 180. The second external device 180 may be monitored by the patient's physician or other health care professional, and upon receiving the warning, the physician can act to remedy the failure of the patient to recharge the battery and the resultant diminishing charge of the battery.

As referred to in the previous paragraph, information relating to the battery status may be transmitted first to the first external device 170, and then at a later time to the second external device 180. In a particular example, the battery status relates to the charge remaining in the rechargeable battery. The telemetry circuit 220 can be configured to transmit information relating to the remaining charge in the battery at the first time to the first external device in response to a first charge level. For example, if the battery has 25% of its charge remaining (i.e., 25% of a fully charged state), that can serve as a threshold to trigger the transmission of battery status information to the first external device 170. Other levels of remaining charge can be chosen depending on numerous factors including the type or device, the individual patient, the functions performed by the device, the type of battery in the device, etc. Then, if the battery 210 is not recharged before it reaches a second remaining charge level, for example if the charge level of the battery falls to 10%, a signal can be transmitted to the second external device 180. The second external device 180, which may be monitored by the patient's physician, can then take action in response to the failure of the patient to take steps to recharge the battery in the implantable device.

In a similar example, the warnings transmitted to the first external device 170 or the second external device 180 can be based on a combination of the remaining charge of the battery and the rate of discharge of the rechargeable battery. Therefore, if the control circuit 205 and the battery monitoring circuit 215 sense a high rate of discharge in the battery 210, a warning can be transmitted when the battery has a comparatively higher level of charge remaining than in circumstances when the rate of discharge of the battery is not as great. For example, if the level of charge remaining in the battery reaches a 50% level, and the rate of discharge is at a rather high rate, then the transmission of the signal to the first external device 170 can occur at this 50% level. The rate of discharge can be measured by the computation circuit 225. Similarly, if the remaining charge of the battery then decreases to a second level, for example down to a 25% remaining charge, and the rate of discharge is at a certain level, then an alert can be sent to the second external device when the battery reaches this 25% level. When determining the remaining charge of the battery, one or more of the computation circuits 172, 182, and 225 can be configured to compute this remaining charge.

In another example, the telemetry circuit 220 can be configured to recurrently transmit alerts after the remaining charge level in the battery 210 has crossed a certain threshold. For example, the telemetry circuit can transmit a first alert when the remaining charge level in the battery falls to a 25% level. Then, for example, if the charge level thereafter decreases to 15%, the telemetry circuit can be configured to transmit the alert recurrently. These recurrent transmissions can be based on certain charge level decrements, such as transmitting for each 2% decrease in the charge level (or an absolute decrease in the charge level), or the recurrent transmissions can be based on time, such as recurrently transmitting the alerts every hour until the battery is recharged.

In another example, the system 100 can be configured to maintain a recharging history of the rechargeable battery 210. This history can be maintained in the memory circuit 230 of the implantable device 110, the memory circuit 179 of the first external device 170, or the memory circuit 189 of the second external device 180. When the recharging history is stored in the memory circuit 230 of the device 110, the control circuit 205 monitors the battery recharging circuit 240 and the rechargeable battery 210. When the control circuit initiates recharging of the battery through the recharging circuit 240, the control circuit thereafter monitors the recharging process, and if the rechargeable battery crosses a threshold, for example it becomes 90% recharged, then the control circuit treats this as a successful recharge and writes a record to the recharging history in the memory circuit 230. When the recharging history is maintained in the first or second external device, the control circuit 205 monitors the recharging process, and when the charge level in the battery crosses the threshold, the control circuit causes the telemetry circuit 220 to transmit an indication of the recharging to the first or second external device for storage in the recharging history maintained in those devices. A recharging history in the second external device can be used by a physician or other caregiver to monitor a patient's compliance with the recharging of the battery in the implanted device.

The transmission of information relating to the status of the battery 210, or any other data or information, can be transmitted from the implantable device 110 to the first or second external devices via a radio frequency (RF) signal. In an example, an implantable medical device can be configured to transmit one or more of an Industrial, Scientific and Medical (ISM) signal, a Medical Implant Communication Service (MICS) signal, a BLUETOOTH® signal, and a Short Range Device (SRD) signal.

In an example, the battery monitoring circuit 215 monitors the charge level of the battery 210, and triggers an alert when the charge level of the battery falls below a threshold. Such an alert can be transmitted by the telemetry circuit 220 to the first external device 170 or the second external device 180. When transmitted to the first or second external device, the alert can be an audible alert, a vibration alert, or a visual alert (displayed on the display units 176 or 186). A vibration alert can also be programmed into the implantable device itself.

In an example, the implantable medical device 110 is configured such that the telemetry circuit 220 is limited in the transmission of information relating to the battery status (or other data or information) when the level of charge remaining in the battery crosses a threshold. Other functionalities of the implantable medical device may also be disabled under a low charge condition (such as rate-responsiveness for example). This feature prevents the implantable device 110 from unduly wasting the remaining charge on the battery with transmissions of information to the external devices, thereby conserving the diminishing charge in the battery for the vital functions of the implantable device.

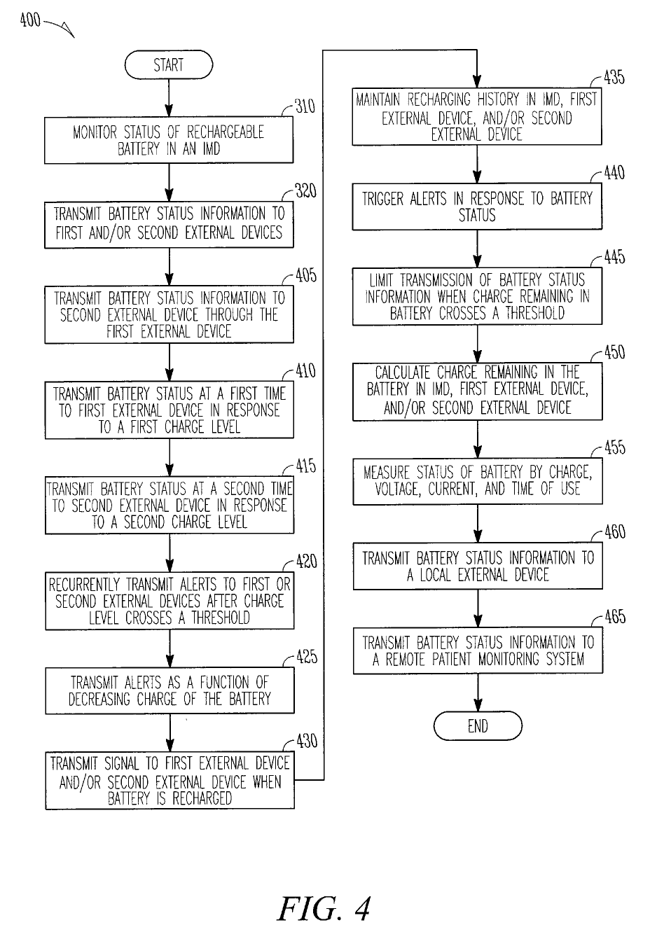

FIGS. 3 and 4 illustrate example processes 300 and 400 respectively for monitoring the battery status of an implantable medical device, and in an example, the level of charge remaining in that battery. In FIG. 3, at 310, a battery status of a rechargeable battery in an implantable medical device is monitored. At 320, information relating to the battery status of the rechargeable battery is transmitted to a first external device at a first time and to a second external device at a second time. In this process 300, the second external device is of a different type than the first external device.

FIG. 4 illustrates another example process 400 for monitoring the charge state of a battery of an implantable medical device. Not all the steps of process 400 need be executed in any particular example, and the steps of a particular example need not be executed in the order outlined in FIG. 4. The process 400 includes the steps 310 and 320 of process 300. At 405, the information relating to the battery status of the rechargeable battery is transmitted to the second external device through the first external device. At 410, the information relating to the battery status is transmitted at a first time to the first external device in response to a level of remaining charge in the rechargeable battery, and at 415 the information relating to the remaining charge in the battery or other battery status is transmitted at a second time to the second external device in response to a second level of remaining charge in the rechargeable battery. In steps 410 and 415, the remaining charge levels at the first and second times are different.

At 420, alerts are recurrently transmitted to the first or second devices after the remaining charge in the rechargeable battery has crossed a threshold. The threshold normally signals a level of charge that is approaching a point at which operation of the device can be imperiled, and the recurrent transmissions notify the patient or doctor of that potentially dangerous charge level. Similarly, at 425, alerts are transmitted as a function of a decreasing charge of the rechargeable battery. At 430, a signal is transmitted to one or more of the first external device and the second external device when the rechargeable battery is recharged, and at 435, a record is maintained in one or more of the implantable device, the first external device, and the second external device regarding a recharging history of the rechargeable battery.

At 440, one or more of an audible alert, a visual alert, or a vibration alert is triggered in response to the information relating to the battery status. At 445, the transmission of the information relating to the battery status is limited when the remaining charge in the rechargeable battery crosses a threshold. This conserves the dwindling battery energy for vital device functions when the charge remaining in the battery becomes too low. At 450, the remaining charge in the rechargeable battery is calculated in one or more of the implantable medical device, the first external device, and the second external device. At 455, the battery status is monitored by measuring one or more of a charge associated with the rechargeable battery, a voltage output of the rechargeable battery, a current flow through the rechargeable battery, and a time that the battery has been used since an initial charging or recharging of the rechargeable battery. At 460, the information relating to the battery status of the rechargeable battery is transmitted to a first external device at a first time that includes a local external device to which a patient or a patient's local caregiver has access. At 465, the information relating to the battery status of the rechargeable battery is transmitted to a second external device at a second time that includes a remote patient monitoring system that is accessible by one or more of a physician and a plurality of implantable medical devices.

In the foregoing detailed description, various features are grouped together in one or more examples or examples for the purpose of streamlining the disclosure. This method of disclosure is not to be interpreted as reflecting an intention that the claimed examples of the invention require more features than are expressly recited in each claim. Rather, as the following claims reflect, inventive subject matter lies in less than all features of a single disclosed example. Thus the following claims are hereby incorporated into the detailed description of examples of the invention, with each claim standing on its own as a separate example. It is understood that the above description is intended to be illustrative, and not restrictive. It is intended to cover all alternatives, modifications and equivalents as may be included within the scope of the invention as defined in the appended claims. Many other examples will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein,” respectively. Moreover, the terms “first,” “second,” and “third,” etc., are used merely as labels, and are not intended to impose numerical requirements on their objects.

As used in this disclosure, the term “circuit” is broadly meant to refer to hardware, software, or a combination of hardware and software. That is, a particular function may be implemented in specialized circuits, in software executing on general processor circuits, and/or a combination of specialized circuits, generalized circuits, and software.

The abstract is provided to comply with 37 C.F.R. 1.72(b) to allow a reader to quickly ascertain the nature and gist of the technical disclosure. The Abstract is submitted with the understanding that it will not be used to interpret or limit the scope or meaning of the claims.

1. An apparatus comprising:
an implantable medical device comprising:
a control circuit;
a rechargeable battery, the rechargeable battery coupled to the control circuit;
a battery monitoring circuit, the battery monitoring circuit coupled to one or more of the rechargeable battery and the control circuit; and
a telemetry circuit, coupled to the control circuit, wherein the telemetry circuit is configured to transmit a battery status of the rechargeable battery to a first external device at a first time and to a second external device, of a different type than the first external device, at a second time.

2. The apparatus of claim 1, wherein the telemetry circuit is configured to transmit a battery status of the rechargeable battery to the first external device at a first time, and to the second external device via the first external device at a second time.

3. The apparatus of claim 1, further comprising the first and second external devices.

4. The apparatus of claim 3, wherein the first external device includes a local external programmer device, and wherein the second external device includes a remote external patient management device configured to manage implantable medical devices associated with different patients.

5. The apparatus of claim 3, wherein one or more of the implantable medical device, the first external device, and the second external device comprise a computation circuit configured to perform a computation relating to the battery status that is carried out in one or more of the implantable medical device, the first external device, and the second external device.

6. The apparatus of claim 5, comprising an external display configured to display the battery status as a graphic, wherein the external display is associated with a user interface of one or more of the first or second external devices.

7. The apparatus of claim 5, wherein the computation circuit is configured to perform a computation of a remaining charge of the rechargeable battery.

8. The apparatus of claim 1, wherein the battery status relates to a remaining charge of the rechargeable battery, and wherein the telemetry circuit is configured to transmit information relating to the remaining charge at the first time to the first external device in response to a first level of remaining charge, and further wherein the telemetry circuit transmits the information relating to the remaining charge at the second time to the second external device in response to a second level of remaining charge that is different from the first level of remaining charge.

9. The apparatus of claim 1, wherein the battery status relates to a remaining charge of the rechargeable battery and a rate of discharge of the rechargeable battery, and wherein the telemetry circuit is configured to transmit information relating to the remaining charge and the rate of discharge at the first time to the first external device in response to a first level of remaining charge and a first rate of discharge, and further wherein the telemetry circuit transmits the information relating to the remaining charge and the rate of discharge at the second time to the second external device in response to a second level of remaining charge and a second rate of discharge that are different from the first level of remaining charge and the first rate of discharge.

10. The apparatus of claim 1, wherein the telemetry circuit is configured to recurrently transmit alerts after a level of remaining charge has crossed a certain threshold.

11. The apparatus of claim 1, wherein the telemetry circuit is configured to transmit alerts as a function of decreasing charge levels of the rechargeable battery.

12. The apparatus of claim 1, wherein at least one of the implantable medical device, the first external device, and the second external device is configured to maintain a history of recharging of the rechargeable battery.

13. The apparatus of claim 1, wherein the telemetry circuit is configured to transmit the battery status to one or more of the first external device and the second external device using an RF signal such as an Industrial, Scientific and Medical (ISM) signal, a Medical Implant Communication Service (MICS) signal, a BLUETOOTH® signal, and a Short Range Device (SRD) signal.

14. The apparatus of claim 1, wherein the battery status triggers an alert by one or more of the first external device and the second external device, the alert including one or more of an audible alert, a visual alert, and a vibration alert.

15. The apparatus of claim 1, wherein the implantable medical device is configured such that the telemetry circuit is limited in the transmission of the battery status when a level of remaining charge of the rechargeable battery crosses a threshold.

16. The apparatus of claim 1, wherein information relating to the battery status comprises one or more of a measurement of a charge associated with the rechargeable battery, a voltage output of the rechargeable battery, a current flow through the rechargeable battery, and a time of use of the rechargeable battery since initial charging or a recharging.

17. A process comprising:
monitoring a battery status of a rechargeable battery in an implantable medical device; and
transmitting information relating to the battery status of the rechargeable battery to a first external device at a first time and to a second external device at a second time, wherein the second external device is of a different type than the first external device.

18. The process of claim 17, wherein the transmitting information relating to the battery status of the rechargeable battery to the second external device passes through the first external device.

19. The process of claim 17, comprising:
transmitting the information relating to the battery status at the first time to the first external device in response to a first level of remaining charge of the rechargeable battery; and
transmitting the information relating to the battery status at the second time to the second external device in response to a second level of remaining charge of the rechargeable battery that is different than the first level of remaining charge.

20. The process of claim 17, comprising recurrently transmitting alerts after a level of remaining charge of the rechargeable battery has crossed a threshold.

21. The process of claim 17, comprising transmitting alerts as a function of a decreasing charge of the rechargeable battery.

22. The process of claim 17, comprising:
transmitting a signal to one or more of the first external device and the second external device when the rechargeable battery is recharged; and
maintaining a record in one or more of the implantable device, the first external device, and the second external device regarding a recharging history of the rechargeable battery.

23. The process of claim 17, comprising:
monitoring the battery status by measuring one or more of a charge associated with the rechargeable battery, a voltage output of the rechargeable battery, a current flow through the rechargeable battery, and a time that the battery has been used since an initial charging or recharging of the rechargeable battery; and
triggering, in response to the information relating to the battery status, one or more of an audible alert, a visual alert, and a vibration alert.

24. The process of claim 17, comprising:
calculating a level of remaining charge of the rechargeable battery in one or more of the implantable medical device, the first external device, and the second external device; and
limiting the transmission of the information relating to the battery status when a level of remaining charge of the rechargeable battery crosses a threshold.

25. The process of claim 17, wherein transmitting information relating to the battery status of the rechargeable battery to a first external device at a first time includes transmitting the information to a local external device to which a patient or a patient's local caregiver has access, and wherein transmitting information relating to the battery status of the rechargeable battery to a second external device at a second time includes transmitting the information to a remote patient monitoring system that is accessible by one or more of a physician and a plurality of implantable medical devices.

26. An apparatus comprising:
an implantable medical device comprising:
a control circuit;
a rechargeable battery, the rechargeable battery coupled to the control circuit;
a battery monitoring circuit, the battery monitoring circuit coupled to one or more of the rechargeable battery and the control circuit; and
a telemetry circuit, coupled to the control circuit, wherein the telemetry circuit is configured to transmit a battery status of the rechargeable battery to one or more of a first external device and a second external device, the second external device of a different type than the first external device;
wherein one or more of the first external device and the second external device are configured to trigger an alert at a first time and at a later second time based on the transmitted battery status.
